(19) United States
(12) Patent Application Publication
(10) Pub. No.: US 2026/0256380 A1
(43) Pub. Date: Sep. 3, 2026

(54) THERAPY FOR THE TREATMENT OF DISORDERS IN THE NERVOUS SYSTEM USING BIOMARKERS

(71) Applicant: BIOTRONIK SE & Co. KG, Berlin (DE)

(72) Inventors: Sebastian Schulze-Luckow, Berlin (DE); Andrew B. Kibler, Lake Oswego, OR (US); Koeun Lim, Tucson, AZ (US)

(21) Appl. No.: 19/489,955

(22) PCT Filed: Jun. 4, 2024
(86) PCT No.: PCT/EP2024/065268

§ 371 Date: Dec. 4, 2025

Related U.S. Application Data
(60) Provisional application No. 63/471,640, filed on Jun. 7, 2023.

(30) Foreign Application Priority Data
Jul. 4, 2023 (EP) 23183417.7

(51) Int. Cl. A61B 5/11; A61B 5/00; A61B 5/372; A61N 1/36

(52) U.S. Cl. CPC A61B 5/1101; A61B 5/372; A61B 5/4082; A61B 5/4836; A61B 5/681; A61N 1/36067; A61N 1/36139

(57) ABSTRACT

A method includes: acquiring, with a medical device, at least a first electrical signal from a brain or a spinal cord of a patient; generating, with a wearable device worn by the patient, at least a second signal relating to the patient; determining whether at least a first predetermined biomarker is present in at least one signal; determining whether at least a second predetermined biomarker is present in at least one signal; determining whether the first predetermined biomarker is present in the at least one first electrical signal and the second predetermined biomarker is present in the at least one second signal; based on whether or not the first predetermined biomarker is present and whether or not the second predetermined biomarker is present and whether or not the first and the second predetermined biomarkers are present, either adjusting at least one therapy parameter, or determining a patient state.

The present invention relates to a method and a system for detecting biomarkers relating to tremors.

Typical symptoms of diseases like Parkinson's are tremors. A tremor is an involuntary, often rhythmic, muscle contraction and relaxation involving back and forth movements of one or more body parts. It is one of the most common involuntary movements, and frequently occurs in the hands of a patient, but it can also affect other body parts such as arms, eyes, face, head, vocal folds, trunk, and legs.

Known solutions for diagnosing/treating tremors require visiting a physician for clinical presentation, magnetic resonance therapy of the brain after medical diagnosis, and measurement of muscle activity by means of electrodes in the clinic.

However, all of the above measures typically involve visiting a physician. The typical population of patients is regularly older than 65 years, the repeated visit of the doctor is necessary for the diagnosis of this population. Thus, particularly, only a temporary measurement (time window) is provided, not a permanent monitoring, so that an insufficient diagnosis is a possible outcome. Furthermore, the diagnosis depends on the experience of the physician, wherein such diagnosis usually confirms that a tremor is either present or not, and may describe the quality of the tremor as severe or mild-but will regularly not be given as objectively measured value. Therefore, existing solutions are often partially unnecessary, expensive, time consuming, i.e. they tie up personnel, not comparable to the advance of the state of knowledge, and possibly prone to error as the physician evaluates.

Therefore, based on the above, the problem to be solved by the present invention is to provide a method and a system that allow to detect involuntary body movements such as tremors.

This problem is solved by a method having the features of claim 1 as well as by a system having the features of claim 30, and by a system having the feature claim 51. Preferred embodiments of these aspects of the present invention are stated in the corresponding dependent claims and are described below.

According to claim 1 a method is disclosed, the method comprising the steps of:
acquiring, with a medical device, at least a first electrical signal from a brain or a spinal cord of a patient, the first electrical signal being indicative of a tremor of the patient;
generating, with a wearable device worn by the patient, at least a second signal associated with the patient, the second signal being particularly indicative of a tremor of the patient when the patient is experiencing a tremor;
determining whether at least a first predetermined biomarker is present in at least one signal; determining whether at least a second predetermined biomarker is present in at least one signal;
determining whether the first predetermined biomarker is present in the at least one first electrical signal and the second predetermined biomarker is present in the at least one second signal;
based on whether or not the first predetermined biomarker is present and whether or not the second predetermined biomarker is present and whether or not the first and the second predetermined biomarkers are present, either adjusting at least one therapy parameter, or determining a patient state.

Preferably, the medical device is an implantable medical device, such as a brain pacemaker or neurostimulator (e.g. for spinal cord stimulation).

Particularly an electrical signal from the spinal cord can be an evoked compound action potential (eCAP), which is the result of summation of many action potentials from the individual axons in the nerve trunk. The eCAP carries information regarding the electrical activity of the nerves, and can be used in spinal cord stimulation to implement a closed stimulation loop for pain treatment.

Particularly, this can mean that first predetermined biomarker is present in the first electrical signal and the second biomarker in a further electrical signal. It can also mean that both of these markers are present in the first electrical signal or in the second signal etc. Particularly, it can occur that the first predetermined biomarker is present in the first electrical signal and the second predetermined biomarker is present in the second signal.

Furthermore, according to a preferred embodiment of the method according to the present invention, the wearable device is one of: a smartwatch, a smart necklace, a smart anklet. Particularly, each of these smart devices is characterized in that it comprises a user interface for displaying information to a user (e.g. patient) and receiving input from the user, and a processor configured to execute a computer program that conducts acquiring the second signal, and particularly to preprocess and/or transmit said second signal to the at least one processor. The latter can be located in a remote service center, but can also be located in an external device (e.g. in a mobile device, in a patient device, in a programmer).

Furthermore, particularly, the second signal generated by the wearable device preferably represents a movement of a body part of the patient wearing the wearable device. Particularly said movement of the body part can be a tremble of one of: a hand of the patient wearing the wearable device, a head of the patient wearing the wearable device, a foot of the patient wearing the wearable device. However, other second signals relating to physiological parameters of the patient are also conceivable.

Further, in a preferred embodiment of the method, the first electrical signal is a motor cortex signal and the first predetermined biomarker is a peak in the motor cortex signal.

Furthermore, according to a preferred embodiment of the method, the first predetermined biomarker and/or the second predetermined biomarker are patient specific.

In some examples, information regarding one or more patient specific biomarkers may allow for an enhanced ability to provide individualized therapy.

In a preferred embodiment of the method, the first and/or the second predetermined biomarker is a peak with an amplitude above a predetermined threshold, or a lack of a peak with an amplitude above a predetermined threshold. Such a peak is also denoted as a spike. Additionally, the first and/or second biomarker may be one of: a peak or series of peaks of a compound action potential signal, preferably an evoked action potential signal from the central nervous system, preferably of a peripheral nerve or spinal cord. In particular, the first and/or second biomarker is at least one of the amplitude of the peak, the timing of the peak, or the absolute area traced by the signal during the time a peak is expected by integration of the area of the compound action potential signal within a period of time, e.g. of up to 10 milliseconds. Multiple peaks may be detected, and the absence of a detected peak within a specified time, typically 20 milliseconds or less after a stimulation pulse may also be a specific biomarker. Biomarkers to detect patient stress and health are also included in a preferred embodiment, covering: resting heart rate, daily heart rate, daily heart rate variability, and changes in arterial pulse wave velocity. These may be measured by an implantable medical device or wearable system component such as a smartwatch, smart phone, external medical device, or patch. Additional preferred include body temperature as measured from an implantable device, and body position and activity signals which are processed to detect: a fall (sudden spike of accelerometer signal) and decrease or increase of activity compared to a rolling average baseline of activity.

Further, according to a preferred embodiment of the method, in case the first predetermined biomarker is present and/or in case the second predetermined biomarker is present and/or in case the first and the second predetermined biomarkers are present, an alert is automatically sent to a remote facility, such as a service center.

According to a preferred embodiment of the method, said patient state is one of: a posture of the patient, a temperature of the patient, a value of a likelihood whether the tremor episode is low or high.

The patient state can further be one of the following states: an unmedicated state where the patient is not receiving any therapy and is displaying symptoms of PD such as bradykinesia, rigidity, dystonia, or tremor; an equilibrium state where the patient's response to therapy includes PD symptoms under control without the presence of side effects; and a side effects state, where the patient is experiencing side effects from over medication such as dyskinesia.

According to a preferred embodiment of the method, the at least one therapy parameter is one of: an amplitude of an electrical stimulation to be applied to the patient, a frequency of an electrical stimulation to be applied to the patient, a duration of an electrical stimulation to be applied to the patient, a type of electrical stimulation to be applied to the patient, a number of succeeding electrical stimulations to be applied to the patient.

According to a preferred embodiment of the method, determining whether at least a first predetermined biomarker is present in the at least one signal comprises determining whether the first predetermined biomarker is present in the first electrical signal acquired from a motor cortex of the brain of the patient, and wherein determining whether at least a second predetermined biomarker is present in the at least one signal comprises determining whether the second predetermined biomarker is present in a second signal generated by the wearable device.

According to a preferred embodiment of the method, a patient state is determined, and further comprising adjusting at least one therapy parameter based on the determined patient state.

According to a preferred embodiment of the method, adjusting the at least one therapy parameter comprises setting a lower bound for the therapy parameter based on the presence of the first predetermined biomarker and setting an upper bound for the therapy parameter based on the presence of the second predetermined biomarker.

According to a preferred embodiment of the method, at least one therapy parameter is adjusted, and wherein adjusting the at least one therapy parameter comprises adjusting a frequency of electrical stimulation. In a preferred embodiment of the method, adjusting a frequency of electrical stimulation comprises adjusting the frequency to at least one of: a frequency of at least one of the first predetermined biomarker and the second predetermined biomarker; approximately 55-65 Hz; or approximately 120-140 Hz.

According to a preferred embodiment of the method, the method further comprises delivering electrical stimulation to a patient at the adjusted frequency.

Further, in a preferred embodiment of the method, the latter further comprises delivering electrical simulation to a subthalamic nucleus (STN) of the brain of the patient at the adjusted frequency.

According to a preferred embodiment of the method, the adjusted frequency is between approximately 120 Hz and 140 Hz.

According to a preferred embodiment of the method, the adjusted frequency is between approximately 55 and 65 Hz.

According to a preferred embodiment of the method, the adjusted frequency is approximately equal to the frequency of one of the first predetermined biomarker or the second predetermined biomarker.

According to a preferred embodiment of the method, adjusting at least one therapy parameter comprises delivering electrical stimulation at alternating frequencies. In a preferred embodiment of the method, the alternating frequencies are approximately 60 Hz, and approximately 130 Hz.

Furthermore, according to a preferred embodiment of the method, determining whether at least a first predetermined biomarker is present in the at least one signal comprises determining whether the first predetermined biomarker is present in the first electrical signal acquired from a motor cortex of the brain of the patient.

According to a preferred embodiment of the method, the first predetermined biomarker is one of: a low beta band biomarker, a high beta band biomarker, a gamma band biomarker.

According to a preferred embodiment of the method, determining whether at least a second predetermined biomarker is present in the at least one signal comprises determining whether the second predetermined biomarker is present in a further signal acquired from a subthalamic nucleus (STN).

Further, according to a preferred embodiment of the method, the second predetermined biomarker is one of: a beta band biomarker, a gamma band biomarker.

According to a preferred embodiment of the method, determining whether at least a first predetermined biomarker is present in the at least one signal comprises determining whether the first predetermined biomarker is present in the first electrical signal acquired from a motor cortex of the brain of the patient, and wherein determining whether at least a second predetermined biomarker is present in the at least one signal comprises determining whether the second predetermined biomarker is present in a further electrical signal acquired from a subthalamic nucleus (STN). For instance, a biomarker which can be identified in the signals obtained by a wearable device can be accelerometer data showing amplitude peaks with a characteristic frequency for tremor.

According to a preferred embodiment of the method, at least one therapy parameter is adjusted, and the method further comprises: acquiring an updated first electrical signal from the motor cortex; acquiring an updated second electrical signal from the STN; determining whether at least the first predetermined biomarker is present in the updated first electrical signal; determining whether at least the second predetermined biomarker is present in the updated second signal; adjusting the therapy parameter based on whether or not the first predetermined biomarker is present and whether or not the second predetermined biomarker is present; analyzing the efficacy of the adjusted therapy parameter based on whether the first predetermined biomarker is present in the updated first electrical signal or second predetermined biomarker are present in the updated second signal; and readjusting the adjusted therapy parameter based on the efficacy of the adjusted therapy parameter.

According to a preferred embodiment of the method, the first predetermined biomarker and the second predetermined biomarker are patient specific.

Furthermore, according to a preferred embodiment of the method, the patient state is dyskinesia.

According to yet another preferred embodiment of the method, the first predetermined biomarker and the second predetermined biomarker are disease specific. Further, according to a preferred embodiment of the method, the disease is Parkinson's disease.

According to yet another aspect of the present invention a system is disclosed, the system comprising:
an implantable medical device configured to be implanted into a patient;
a wearable device configured to be worn by the patient;
at least one processor;
at least a first electrode in communication with the implantable medical device, the first electrode configured to acquire at least a first electrical signal from a brain of a patient, the first electrical signal being indicative of a tremor of the patient when the patient is experiencing tremor; wherein the wearable device is configured to generate a second signal associated with the patient, the second signal being indicative of a tremor of the patient when the patient is experiencing tremor; and wherein the processor is configured to: determine whether at least a first predetermined biomarker is present in at least one signal; whether at least a second predetermined biomarker is present in at least one signal; whether the first predetermined biomarker is present in the at least one first electrical signal and the second predetermined biomarker is present in the at least one second signal; and
based on whether or not the first predetermined biomarker is present and whether or not the second predetermined biomarker is present and whether or not the first and the second predetermined biomarkers are present, either adjusting at least one therapy parameter, or determining a patient state.

In a preferred embodiment of the system, the first electrical signal is a motor cortex signal and the first predetermined biomarker is a peak in the motor cortex signal.

Further, in a preferred embodiment of the system, the first predetermined biomarker and/or the second predetermined biomarker are patient specific.

Further, according to a preferred embodiment of the system, the first and/or the second predetermined biomarker is a peak with an amplitude above a predetermined threshold.

According to yet another preferred embodiment, the system, particularly the at least one processor and/or the medical device and/or the wearable device, is configured to send an alert to a remote facility (particularly a service center) when the first predetermined biomarker is present and/or in case the second predetermined biomarker is present and/or in case the first and the second predetermined biomarkers are present.

According to yet another preferred embodiment of the system, the at least one processor is configured to determine a patient state and adjust at least one therapy parameter based on the determined patient state.

Furthermore, according to a preferred embodiment of the system, the processor is further configured to set a lower bound for the therapy parameter based on the presence of the first predetermined biomarker and set an upper bound for the therapy parameter based on the presence of the second predetermined biomarker.

Furthermore, according to a preferred embodiment of the system, at least one therapy parameter is adjusted, and wherein the at least one processor is further configured to adjust a frequency of electrical stimulation. In a preferred embodiment of the system, the at least one processor is further configured to adjust the frequency to at least one of: a frequency of at least one of the first predetermined biomarker and the second predetermined biomarker; approximately 55 Hz to 65 Hz; or approximately 120 Hz to 140 Hz. Preferably, according to a preferred embodiment, the medical device is configured to deliver electrical stimulation to the patient at the adjusted frequency. Furthermore, in a preferred embodiment of the system, the medical device is configured to deliver electrical stimulation to a subthalamic nucleus (STN) of the brain of the patient at the adjusted frequency via the first electrode. In a preferred embodiment, the adjusted frequency is between approximately 120 Hz and 140 Hz. Preferably, the adjusted frequency is between approximately 55 Hz and 65 Hz. Further, according to a preferred embodiment of the system, the adjusted frequency is approximately equal to the frequency of one of the first predetermined biomarker or the second predetermined biomarker.

In a preferred embodiment of the system, adjusting at least one therapy parameter comprises delivering electrical stimulation at alternating frequencies. According to a preferred embodiment of the system, the alternating frequencies are approximately 60 Hz, and approximately 130 Hz.

According to a preferred embodiment of the system, the first electrode is configured to acquire a first electrical signal form a motor cortex of the brain of the patient, and wherein the processor is further configured to determine whether the first predetermined biomarker is present in the first electrical signal.

Further, according to yet another preferred embodiment of the system, the system comprises a second electrode in communication with the implantable medical device, the second electrode configured to acquire a further electrical signal from a subthalamic nucleus (STN) of the brain of the patient, and wherein the at least one processor is further configured to determine whether the second predetermined biomarker is present in the further electrical signal acquired from the STN.

Furthermore, according to a preferred embodiment of the system, the first predetermined biomarker is one of: a low beta band biomarker, a high beta band biomarker, and a gamma band biomarker.

According to a preferred embodiment of the system, the second predetermined biomarker is one of a beta band biomarker, and a gamma band biomarker.

According to yet another preferred embodiment of the system, the first electrode is further configured to acquire an updated first electrical signal from the motor cortex; the second electrode is further configured to acquire an updated further electrical signal from the STN; and wherein the processor is further configured to:
determine whether at least the first predetermined biomarker is present in the updated first electrical signal;
determine whether at least the second predetermined biomarker is present in the updated second signal;
analyze the efficacy of the adjusted therapy parameter based on whether or not the first predetermined biomarker is present and whether or not the second predetermined biomarker is present; and
readjust the adjusted therapy parameter based on the efficacy of the adjusted therapy parameter.

According to a preferred embodiment of the system, the first predetermined biomarker and the second predetermined biomarker are patient specific.

Furthermore, according to a preferred embodiment of the method, the patient state is dyskinesia.

According to a preferred embodiment of the method, the first predetermined biomarker and the second predetermined biomarker are disease specific. In a preferred embodiment of the system, the disease is Parkinson's disease.

Furthermore, according to yet another aspect of the present invention, a system is disclosed, wherein the system comprises means for acquiring, with a medical device, at least one electrical signal from a brain of a patient; means for generating, with a wearable device, at least one second signal relating to the patient;
means for determining whether at least a first predetermined biomarker is present in the at least one signal; means for determining whether at least a second predetermined biomarker is present in at least one signal; means for determining whether the first predetermined biomarker is present in the at least one first electrical signal and the second predetermined biomarker is present in the at least one second signal;
and either means for adjusting at least one therapy parameter based on whether or not the first predetermined biomarker is present and whether or not the second predetermined biomarker is present and whether or not the first and the second predetermined biomarker are present, or means for determining a patient state based on whether or not the first predetermined biomarker is present and whether or not the second predetermined biomarker is present and whether or not the first and the second predetermined biomarker are present.

In the following, embodiments of the aspects of the present invention as well as further features and advantages of the present invention are described with reference to the Figure, wherein

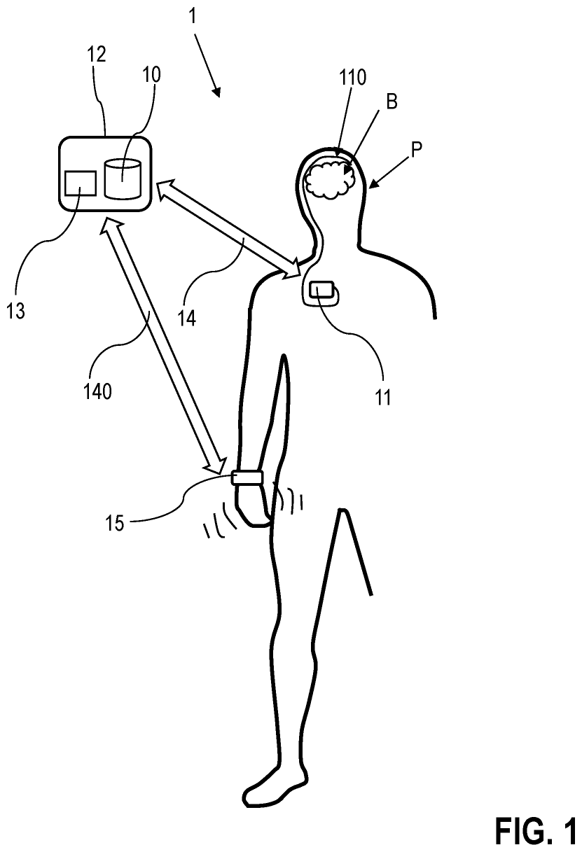

FIG. 1 shows an embodiment of a system and method according to the present invention allowing to detect tremors of a patient based on biomarker detection; wherein preferably the system/method allows controlling therapy based on the biomarker detection and determining patient states.

As indicated in FIG. 1 a system 1 according to a preferred embodiment of the present invention comprises medical device 11, preferably an implantable medical device 11 such as brain pacemaker shown in FIG. 1 for electrically stimulating a brain B of the patient P. Alternatively, the implantable medical device 11 can also be a neurostimulator for stimulating a spinal cord of the patient P. Furthermore, preferably, the system 1 comprises a wearable device 15 configured to be worn by the patient P. In a preferred embodiment the wearable device 15 is a smartwatch as described herein, but may also be another suitable wearable device. Furthermore, the system 1 comprises at least one processor 13. The at least one processor can be comprised by the medical device 11, by the wearable device 15, or by an external device 12 such as a mobile device (e.g. a patient device, a programmer, etc.) or a remote computer system 12 that may be located in a service center. It is also conceivable that the system 1 comprises several processors. Particularly, computation can be distributed between medical device 11, wearable device 15 and external device 12.

Furthermore, the medical device 11 and the wearable device 15 are each preferably configured to communicate with the external device or computer system 12 via a wireless (e.g. radio-based) communication 14, 140 that can involve at least one further device and/or at least one communication network. Particularly, the medical device 11 and the wearable device 15 can each comprise communication circuitry that may include one or more processors, memory, wireless radios, antennae, transmitters, receivers, modulation and demodulation circuitry, filters, amplifiers or the like for radio frequency communication with external devices such as external device 12 or external computer system 12. Furthermore, particularly in case of an external computer system 12 in a service center, the respective wireless communication 14, 140 can be performed via a further intermediate external device (not shown) such as a mobile device (e.g. external programmer, external patient device etc.). The medical device 11 and the wearable device 15 can be configured to use the respective wireless communication 14, 140 to receive data from external device/computer system 12 and/or send data to the external device/computer system 12.

Furthermore, the at least one processor 13 can be configured to apply artificial intelligence (AI) techniques to analyze the acquired (electrical) signals. Such AI techniques may include deep learning or other machine learning techniques. Neural network algorithms are one example of deep learning algorithms/models. The deep learning model/algorithm can be carried out on processor 13, but may also be carried out, either partially or completely, on other devices that can receive the necessary input data from the medical device 11 and wearable device 15. Such input data may be gathered separately and provided to the deep learning model which then processes actual (electrical) signals received from a patient to detect tremors.

Furthermore, the system 1, particularly the medical device 11, comprises at least a first electrode 110, wherein the first electrode 110 configured to acquire at least a first electrical signal from a brain B or spinal cord (not shown) of a patient, wherein the first electrical signal is indicative of a tremor of the patient when the patient is experiencing a tremor. Further, the wearable device 15 is configured to generate a second signal from the patient P that is indicative of an occurring tremor, too. The second signal can be an electrical signal from an acceleration sensor of the wearable device 15/smartwatch. However, the wearable device 15 can also be configured to acquire/generate other signals relating to the patient that are indicative of tremors.

Particularly, the at least one processor 13 is configured to determine whether at least a first predetermined biomarker is present in at least one signal; whether at least a second predetermined biomarker is present in at least one signal; whether the first predetermined biomarker is present in the at least one first electrical signal and the second predetermined biomarker is present in the at least one second signal; and based on whether or not the first predetermined biomarker is present and whether or not the second predetermined biomarker is present and whether or not the first and the second predetermined biomarkers are present, either adjusting at least one therapy parameter, or determining a patient state.

The systems and methods according to the present invention can use sensed brain (or spinal cord) activity to determine whether one or more disease state biomarkers are present. Brain activity may be recorded as electrical signal, for example, in the form of local field potential (LFP) and/or electroencepha logram (EEG) or electrocortieogram (ECOG) signals sensed e.g. by the implantable medical or external device 11, 12. On the other hand, electrical stimuli can be used to affect brain activity. Gamma frequency band oscillations, e.g., between about 35 Hertz (Hz) and about 120 Hz or more, in the central nervous system (CNS), recorded e.g. using LFP and EEG, are associated with normal information processing in movement and sensory structures. Beta frequency band oscillations between about 8 Hz and about 35 Hz, are associated with dysfunctions of CNS circuits that control behavioral movements and cognitive states. Higher frequency stimulation, e.g., about 130 Hz, of subcortical brain areas involved with movement can reduce tremor relating to Parkinson's disease.

Particularly, the first or second predetermined biomarker can in principle be a physiological feature in a signal, such as one of; a peak, a spike (i.e. a peak exceeding a defined threshold), a sequence of peaks and/or spikes, a waveform, a waveform comprising certain characteristics (e.g. wavelength, amplitude), a sequence of waveforms, a pause (e.g. in a signal).

Based on such biomarkers, tremors can be identified in said first electrical signal. Likewise, in a signal of a wearable device, biomarkers can be used to detect movements of the patient P corresponding to tremor which leads to a signal being indicative of the movement of a hand of the person showing accelerations characteristic of a tremor.

In general, such biomarkers can be found by inspecting the acquired/generated signals during an episode of tremor and comparing them to signals in the absence of tremor.

Electrical brain (or spinal cord) signals from patients suffering from Parkinson's disease include several biomarkers that may be used to indicate when adjustments to patient treatment may be beneficial to keep a patient within the therapeutic window. Electrical brain signals may be collected from at least one of: the patient's motor cortex, zona incerta (Zi), subthalmic nucleus (STN), basal ganglia, cerebellum, pedunculopontine nucleus, red nucleus, or lateral globus pallidus. The electrical signals from the motor cortex can be collected from the primary motor cortex (M1), the premotor cortex, the supplementary motor area (SMA), the posterior parietal cortex, or the primary somatosensory cortex. One or more biomarkers may be found in the electrical signals collected from each region of the brain.

An LFP of a patient's STN may also include specific frequency signatures, or biomarkers regarding Parkinson's disease. For example, the LFP of a patient prior to receiving medication may include a peak within the beta band that subsides as the concentration of medication increases. As the medication takes effect, the beta peak shifts to a higher frequency range. In addition, when over medication has occurred a small peak shows up within the gamma range. These biomarkers shown, in the LFP of a patient's motor cortex and ST may be used to create an algorithm which automatically adjusts electrical stimulation and or drug delivery in a patient in order to maintain a patient within the therapeutic window.

Thus, biomarkers may be defined from measured LPF signals from the patient's motor cortex and STN. The biomarkers can be monitored by the implantable medical device 11 or the external device/computer system 12.

In addition, using second signals obtained from the wearable device 15, other biomarker/physiological data can be defined to detect episodes of tremor e.g. in a second signal that is indicative of the acceleration of a hand of the patient on which the wearable device is worn.

All these biomarkers may be used to assess the patient's current disease state. The biomarkers may also be used to serve as an indicator of therapy effectiveness in a device or system. Further, the biomarkers may provide feedback to control the medical device 11, wherein at least one therapy parameter is adjusted to have a desired effect on the delivered therapy.

Particularly, the implantable medical device 11 can be configured to provide electrical stimulation to the brain B of the patient B to modulate one or more biomarkers indicative of Parkinson's disease. The medical implant 11 can in particular continually adjust at least one therapy parameter to maintain the patient within a therapeutic window which provides optimum symptom control with minimal side-effects.

Particularly, an electrical simulation provided by the medical device 11 may include delivering stimulation at approximately 55 Hz to 65 Hz in order to reduce tremor or dyskinesia. Furthermore, particularly, the electrical stimulation may be delivered at approximately 60 Hz to modulate the biomarkers of the patient by reducing low motor cortex beta signals and inducing a higher beta signal. Furthermore, stimulating about 60 Hz may modulates the biomarkers of the patient by reducing low M1 beta signals and inducing a higher beta signal. The medical device 11 can be configured to deliver stimulation at approximately 120 Hz to 140 Hz, thereby reducing bradykinesia and rigidity. In some examples, the medical device may deliver stimulation at approximately 130 Hz. The stimulation at approximately 120 Hz to 140 Hz can modulate the biomarkers of the patient P to reduce the presence of a STN low beta peak. Furthermore, the medical device 11 can be configured to provide burst stimulation with short inter-cycle intervals to decrease power at various frequencies. In some examples, burst stimulation may be provided at approximately the same frequency as the biomarker to be modulated. The burst stimulation can comprise cycling between stimulation being provided, and stimulation being off. Furthermore, the interval between providing stimulation may be short. In some examples, the medical device can alternate between delivery of stimulation at approximately 60 Hz and stimulation at approximately 130 Hz.

Furthermore, the medical device 11 of FIG. 1 can be configured to deliver electrical stimulation therapy to control a patient condition, such as a movement disorder or a neurodegenerative impairment of patient 12. Patient 12 ordinarily will be a human patient. In some cases, however, therapy system 10 may be applied to other mammalian or non-mammalian non-human patients. Particularly, the movement disorder may include various symptoms. Particularly, the movement disorder may be a symptom of Parkinson's diseases (e.g., tremor).

In order to carry out stimulation, the medical device 11 preferably comprises, besides the at least one first electrode 110 for sensing the first electrical signal of the brain B (or spinal cord), at least one further electrode. The electrodes are arranged so as to e.g. sense LFPs and/or deliver electrical stimulation to a tissue site within brain B such as a deep brain site under the dura mater of brain B of patient P. Furthermore, particularly, delivery of electrical stimulation to one or more regions of brain B, such as the subthalamic nucleus (STN), globus pallidus internus (GPi), motor cortex such as M1, or thalamus, can be conducted to manage movement disorders, such as Parkinson's disease/tremor.

Particularly, the medical device 11 can be configured to deliver electrical stimulation to patient P in order to re-establish, or re-induce, gamma frequency band activity within brain B. As mentioned above, gamma frequency band activity may be facilitative of movement and cognitive states, while beta frequency band activity may be inhibitive of movement and perhaps cognitive states. As such, it may be desirable to decrease beta frequency band activity in the brain B and increase gamma frequency band activity in the brain B.

Particularly, beta frequency band activity in the brain B may be decreased and gamma frequency band activity in the brain B may be increased by delivering electrical stimulation to a portion of the brain B at a frequency some predetermined ratio between the detected activity in the gamma band and the frequency of stimulation. In one example, the frequency of the electrical stimulation delivered to the portion of the brain B may be at a constant frequency at some predetermined ratio between the detected activity in the gamma band and the frequency of electrical stimulation. Particularly, medical device 11 can be configured to deliver electrical stimulation to patient 12 at a frequency shown to affect the individual patient's biomarkers.

Furthermore, the at least one processor 13 can be configured to analyze electrical signals from the brain B in order to determine, for example, whether one or more biomarkers is/are present within one or more of the beta frequency band, and the gamma frequency band of the patient's motor cortex or STN. For example, the medical implant 11 may sense first electrical signals of the brain B, measure an amplitude of the sensed first electrical signals, and provide the sensed first electrical signals and measured amplitudes to at least one processor 13. Upon receiving the sensed first electrical signals and measured amplitudes, the at least one processor 13 may analyze the received first electrical signal to determine whether a peak is present in either the motor cortex signal or the STN signal at approximately 20 Hz to 25 Hz, approximately 25 Hz to 35 Hz, and/or at approximately 70 Hz to 75 Hz. Such a peak may be an example for a biomarker. Particularly, the detected peaks can be compared to predetermined biomarkers. Particularly, the amplitude of any peaks presents is also determined. Particularly, the exact location that the at least one processor 13 looks for peaks is determined on a patient specific basis. The exact locations may be stored, e.g., in a storage 10 of the external device or in a storage of the medical implant 11.

Likewise, the at least one processor 13 can be configured to analyze second signals from the wearable device 15 in order to determine, for example, whether one or more biomarkers is/are present within the second signals. A second signal may be indicative of an acceleration of the hand of the patient P und thus indicative of tremors. Here, a sequence of peaks in acceleration can form a biomarker representing a tremor.

After delivering electrical stimulation by medical device 11, or in between electrical stimulation pulses, the medical implant 11 can monitor said first electrical signal(s) from the brain B (or spinal cord) and second electrical signal(s) from the wearable device 15 and let the at least one processor 13 determine whether the delivered electrical stimulation resulted in a modulation of one or more previously detected biomarkers. Based on the current biomarkers, the at least one processor 13 may modify the therapy being provided to patient P. Modification may include adjusting one or more stimulation parameters.

As described above, the at least one therapy parameter to be adjusted is preferably one of: an amplitude of an electrical stimulation to be applied to the patient, a frequency of an electrical stimulation to be applied to the patient, a duration of an electrical stimulation to be applied to the patient, a type of electrical stimulation to be applied to the patient, a number of succeeding electrical stimulations to be applied to the patient.

The method described herein can be conducted with a system having a medical device that has already been implanted in a patient and programmed, or in a clinical setting where a system is being implanted in a patient and programming is being turned on for the first time. Here, in addition to or instead of monitoring biomarkers, the physician/clinician may monitor the motor performance based on the clinical Unified Parkinson's Disease Rating Scale (UPDR8), or similar clinical measure, of a patient. The physician/clinician can use the combination of observed motor performance and measured (electrical) signals from the brain B and wearable 15 to better identify patient specific biomarkers. Furthermore, electrical stimulation can be delivered to patient P in order to monitor the motor performance of patient P in response to receiving the electrical stimulation. By monitoring the motor performance of patient P in response to receiving the electrical stimulation, a physician/clinician can determine effective values of parameters of the electrical stimulation settings that may then be programmed into the medical device 11. Particularly, a physician/clinician may determine the patient's efficacious electrical stimulation in a variety of patient states. The patient states may include an unmedicated state where the patient is not receiving any therapy and is displaying symptoms of Parkinson's disease such as bradykinesia, rigidity, dystonia, or tremor; an equilibrium state where the patient's response to therapy includes Parkinson's symptoms under control without the presence of side effects; and a side effects state, where the patient is experiencing side effects from over medication such as dyskinesia. The determined stimulation settings may be programmed into the medical device 11 or stored in storage 10 for later use.

Furthermore, the at least one processor 13 may determine an appropriate therapy based on detected current biomarkers. Particularly, the determination of appropriate therapy may be made automatically without user intervention. Particularly, the at least one processor 13 may first determine a patient state based on the determined biomarkers, and select therapy-parameters based on the determined patient state. Particularly, a low beta peak in the motor cortex signal may indicate a patient is experiencing dyskinesia from overmedication. In response, the at least one processor 13 can instruct medical device 11 to apply stimulation at approximately 55 Hz to 65 Hz to the STN in order to artificially raise and broaden the motor cortex signal beta peak to between approximately 25 Hz to 35 Hz, thus lowering the presence of side effects from over medication.

In some examples, a low beta peak in the ST signal may indicate that the patient P is experiencing either dystonia or tremors. The at least one processor 13 can then instruct medical device 11 to apply stimulation at approximately 60 Hz to the STN in order to artificially raise and broaden the beta peak to between approximately 25Hz to 35 Hz, thus reducing the presence of tremors. Particularly, the medical device 11 can apply stimulation at approximately 130 Hz in response to low beta peak in the STN signal. Furthermore, particularly, in response to a peak in the gamma frequency in either the STN signal or the motor cortex signal, the at least one processor 13 may instruct the medical device 11 to apply burst stimulation in order to decrease the peaks in the gamma frequency. For example, burst stimulation at approximately the same frequency as the detected peaks may be applied to the STN.

The techniques described herein can be implemented, at least in part, in hardware, software, firmware or any combination thereof. For example, various aspects of the techniques may be implemented within one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, or other devices. The term processor may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable (particularly non-transitory) storage medium such as random access memory (RAM), read-only memory (ROM), non-volatile random access memory (NVRAM), electrically erasable programmable read-only memory (EEPROM), FLASH memory, magnetic media, optical media, or the like. The instructions may be executed to support one or more aspects of the functionality described herein.

1. A method comprising:
acquiring, with a medical device (11), at least a first electrical signal from a brain (B) or a spinal cord of a patient (P);
generating, with a wearable device (15) worn by the patient, at least a second signal relating to the patient (P);
determining whether at least a first predetermined biomarker is present in at least one signal;
determining whether at least a second predetermined biomarker is present in at least one signal;
determining whether the first predetermined biomarker is present in the at least one first electrical signal and the second predetermined biomarker is present in the at least one second signal; and
based on whether or not the first predetermined biomarker is present and whether or not the second predetermined biomarker is present and whether or not the first and the second predetermined biomarkers are present, either:
adjusting at least one therapy parameter, or determining a patient state.

2. The method of claim 1, wherein either:
the first electrical signal is a motor cortex signal and the first predetermined biomarker is a peak or a lack of a peak in the motor cortex signal, or
the first electrical signal is a compound action potential signal and the first predetermined biomarker is at least one of a peak, a lack of a peak in the compound action potential signal or an integration of the area of the compound action potential signal within a period of time.

3. The method of claim 1, wherein one or both of the first predetermined biomarker or the second predetermined biomarker are patient specific.

4. The method according to claim 1, wherein one or both of the first predetermined biomarker or the second predetermined biomarker is a peak with an amplitude above a predetermined threshold.

5. The method according to claim 1, wherein an alert is automatically sent to a remote facility in response to any of the following conditions:
the first predetermined biomarker is present,
the second predetermined biomarker is present, or
the first and the second predetermined biomarkers are present.

6. The method according to claim 1, wherein said state includes one of: a posture of the patient, a temperature of the patient, or a value of a likelihood whether the tremor episode is low or high.

7. The method according to claim 1, wherein the at least one therapy parameter includes one of: an amplitude of an electrical stimulation to be applied to the patient, a duration of an electrical stimulation to be applied to the patient, a type of electrical stimulation to be applied to the patient, or a number of succeeding electrical stimulations to be applied to the patient.

8. The method according to claim 1, wherein determining whether at least a first predetermined biomarker is present in the at least one signal comprises determining whether the first predetermined biomarker is present in the first electrical signal acquired from a motor cortex of the brain of the patient, and wherein determining whether at least a second predetermined biomarker is present in the at least one signal comprises determining whether the second predetermined biomarker is present in the second signal generated by the wearable device (15).

9. A system comprising:
an implantable medical device (11) configured to be implanted into a patient (P);
a wearable device (15) configured to be worn by the patient (P);
at least one processor (13);
at least a first electrode (110) in communication with the implantable medical device (11), the first electrode (110) configured to acquire at least a first electrical signal from a brain (B) or spinal cord of a patient;
wherein the wearable device (15) is configured to generate a second signal relating to the patient (P); and
wherein the at least one processor (13) is configured to:
determine whether at least a first predetermined biomarker is present in at least one signal;
whether at least a second predetermined biomarker is present in at least one signal;
whether the first predetermined biomarker is present in the at least one first electrical signal and the second predetermined biomarker is present in the at least one second signal; and
based on whether or not the first predetermined biomarker is present and whether or not the second predetermined biomarker is present and whether or not the first and the second predetermined biomarkers are present, either:
adjusting at least one therapy parameter, or
determining a patient state.

10. The system of claim 9, wherein the at least one processor (13) is configured to determine a patient state and adjust at least one therapy parameter based on the determined patient state.

11. The system of claim 9, wherein the at least one processor (13) is further configured to set a lower bound for the therapy parameter based on the presence of the first predetermined biomarker and set an upper bound for the therapy parameter based on the presence of the second predetermined biomarker.

12. The system of claim 9, wherein at least one therapy parameter is adjusted, and wherein the at least one processor (13) is further configured to adjust a frequency of electrical stimulation.

13. The system of claim 12, wherein the at least one processor (13) is further configured to adjust the frequency to at least one of: a frequency of at least one of the first predetermined biomarker and the second predetermined biomarker; approximately 55 Hz to 65 Hz; or approximately 120 Hz to 140 Hz.

14. The system of claim 12, wherein the medical device (11) is configured to deliver electrical stimulation to the patient (P) at the adjusted frequency.

15. The system of claim 14, wherein the medical device (11) is configured to deliver electrical stimulation to a subthalamic nucleus (STN) of the brain (B) of the patient (P) at the adjusted frequency via the first electrode (110).
